(19) United States
(12) Patent Application Publication
(10) Pub. No.: US 2009/0163573 A1
(43) Pub. Date: Jun. 25, 2009

(54) COMPOSITIONS AND METHODS FOR ENHANCING PROTEIN ACCRETION IN SKELETAL MUSCLE

(75) Inventors: Marvin A. Heuer, Mississauga (CA); Ken Clement, Mississauga (CA); Michele Molino, Mississauga (CA); Joseph MacDougall, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: MULTI FORMULATIONS LTD., Mississauga (CA)

(21) Appl. No.: 11/962,948

(22) Filed: Dec. 21, 2007

(51) Int. Cl. A61P 21/00; A61K 31/385; A61K 31/195

(52) U.S. Cl. 514/440; 514/563

(57) ABSTRACT

A nutritional composition comprising at least an effective amount of geranylgeranylacetone or derivative of geranygeranylacetone and an effective amount of glutamine or derivative of glutamine, wherein the ingredients act substantially simultaneously to increase protein synthesis and inhibit protein degradation in skeletal muscle, via enhanced expression of heat shock proteins in cells, particularly heat shock protein 72. A method of same is also provided.

### FIELD OF THE INVENTION

The present invention relates to a nutritional composition and method for enhancing protein accretion in cells, particularly in skeletal muscle cells. Specifically, the present invention relates to a composition and method comprising a combination of geranylgeranylacetone and glutamine, which act substantially simultaneously, via differing mechanisms, to increase the expression of molecular chaperones in cells, particularly heat shock proteins in skeletal muscle.

### BACKGROUND OF THE INVENTION

When a mammalian cell is exposed to a sudden elevation in temperature the expression of most cellular proteins is decreased. However, some proteins, specifically heat shock proteins (HSP), show increased levels of expression when cells are subjected to elevated temperatures and other metabolic stresses. Examples of metabolic stresses which elicit elevated expression of heat shock proteins include: decreased glucose availability; increased intercellular calcium levels; and decreased blood flow.

Heat shock proteins function as molecular chaperones to prevent protein aggregation and facilitate the folding of non-native proteins, particularly new peptides emerging from ribosomes. Molecular chaperones recognize non-native proteins, predominantly via exposed hydrophobic residues, and bind selectively to those proteins to form relatively stable complexes. In these complexes, the protein is protected and able to fold into its native form.

Among the many families of heat shock proteins, HSP72, the stress-inducible protein of the HSP70 family, is one of the best known endogenous factors protecting cells against tissue injury. Research of exercise-induced stress response has shown that exercise results in increased expression of HSP72 mRNA and subsequently in HSP72 protein.

Repetitive, forceful muscular contractions, i.e. physical exercise, cause changes in the expression patterns of genes and proteins. These changes can result in muscle adaptations such as muscle atrophy via muscle protein catabolism or muscle hypertrophy via muscle protein accretion. During hypertrophy, numerous nascent proteins are formed. An increase in the presence of molecular chaperones, such as HSP72, will act to enhance the stability of these nascent proteins until they can fold into their native forms.

In situations of enhanced protein turnover, such as the environment in muscle following exercise, it would be advantageous for an individual to have a means of increasing the stability of rapidly forming proteins in order to reduce the catabolism of these new non-native state proteins.

### SUMMARY OF THE INVENTION

The present invention relates to a nutritional composition and method for enhancing protein accretion in cells, particularly in skeletal muscle cells. The nutritional composition, comprising an effective amount of at least geranylgeranylacetone or derivative of geranylgeranylacetone and an effective amount of glutamine or derivative of glutamine acting substantially simultaneously to increase protein synthesis and inhibit protein degradation in skeletal muscle, particularly by enhancing the expression of heat shock protein in skeletal muscle. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional composition and method for enhancing protein accretion in cells, particularly in skeletal muscle cells. The nutritional composition, comprising an effective amount of geranylgeranylacetone or derivative of geranylgeranylacetone and an effective amount of glutamine or derivative of glutamine functioning substantially simultaneously to increase protein synthesis and inhibit protein degradation in skeletal muscle, particularly by enhancing the expression of heat shock protein in skeletal muscle.

A used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’, as disclosed herein, belong to a category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer to any substance that is a food, a part of food, or an extract of food which is suitable for consumption by an individual and provides a physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, softgel capsules, gelcaps and the like, not associated with food.

Extracts suitable for use in the present invention may be produced by extraction methods as are known and accepted in the art such as alcoholic extraction, aqueous extractions, carbon dioxide extractions, for example.

As used herein, the term ‘heat shock protein’ is understood to encompass both proteins that are expressly labeled as such as well as other stress proteins, including homologs of such proteins that are expressed in the absence of stressful conditions. Furthermore, as used herein, the term ‘heat shock protein’ is understood to encompass the mRNA species corresponding to expressly labeled heat shock proteins as well as other stress proteins, which are known to be translated into proteins.

As used herein, the term “heat shock response” is understood to be any biological, chemical or biochemical response that results in the activation of heat shock proteins as is herein defined and described.

Geranylgeranylacetone (GGA)

Geranylgeranylacetone is an acyclic polyisoprenoid that has been used to protect gastric mucosa. GGA has been shown to activate transcription factors, particularly heat shock transcription factor (HSF)-1, which are able to bind to DNA and induce transcription. HSF-1 is normally suppressed since it is typically bound to the C-domain of constitutively active HSP70. GGA is able to bind to the C-domain of the HSP70 thereby causing HSF-1 to dissociate. HSF-1 is now able to undergo trimerization and be translocated to the nucleus, where it binds to the heat shock-responsive element (HSE) in the promoter region of inducible HSP70 (i.e. HSP72) genes.

Recent experiments using cultured mouse skeletal cells, showed that treatment with GGA up-regulated the expression of HSP72, and increased muscular protein content in a dose-dependent manner. Additionally GGA was shown to facilitate the differentiation of myoblasts into myotubules.

Non-differentiated myoblasts, often referred to as satellite cells, are a small population of quiescent muscle precursor cells that occupy a “satellite” position immediately outside of muscle fibers. They are normally maintained in a quiescent state and become activated to fulfill roles of routine maintenance, repair and hypertrophy. Satellite cells are thought to be muscle-specific stem cells which are capable of producing large numbers of differentiated progeny as well as being capable of self-renewal. Such that satellite cells can fulfill their biological role, they must become activated, proliferate, differentiate and fuse to existing muscle cells. In this way, multinucleate muscle fibers are maintained or increased in size in response to stimuli.

It is herein understood by the inventors that inclusion of geranylgeranylacetone or derivatives of geranylgeranylacetone in a nutritional composition, will act to increase the expression of heat shock proteins, particularly HSP72, via directly activating HSF-1. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

Additionally, it is herein understood by the inventors that administration of GGA will have the added benefit of facilitating the differentiation of myoblasts to myofibers. These myofibers fuse with existing muscle cells thereby increasing the size of the muscle cells and ultimately muscle tissue.

As used herein, a serving of the present nutritional composition comprises from about 1 mg to about 300 mg of geranylgeranylacetone or derivatives of geranylgeranylacetone. More preferably, a serving of the present nutritional composition comprises from about 25 mg to about 150 mg of geranylgeranylacetone or derivatives of geranylgeranylacetone. A serving of the present nutritional composition most preferably comprises from about 25 mg to about 75 mg of geranylgeranylacetone or derivatives of geranylgeranylacetone.

Glutamine

Glutamine is the most abundant amino acid found in the body and has important functions as a precursor for the synthesis of other amino acids. Many cells required for immune function rely on Glutamine as a source for energy production.

Physical activity can deplete Glutamine levels, and as such, Glutamine is often considered to be a ‘conditionally essential’ amino acid. A study examining the Glutamine levels of groups involved in several different types of activities or sports found that powerlifters and swimmers had lower Glutamine levels than cyclists and non-athletes, suggesting that high resistance load activities require increased amounts of Glutamine.

Administration of glutamine has been shown to enhance protein expression and inhibit protein degradation in a condition-dependent manner. This regulation of protein turnover has been attributed to glutamine's affect on the expression of heat shock proteins in stressed conditions. Glutamine is capable of increasing the expression of heat shock proteins only in conditions of stress.

A study of the mechanism by which glutamine effects the expression of heat shock proteins has shown that glutamine does not affect the classical pathway of HSF-1 activation. Instead, glutamine specifically modulates the transcriptional regulatory apparatus at the HSP promoter, in a manner that is independent of HSF-1.

A specific derivative of glutamine, a dipeptide of alanine and glutamine i.e. Alanyl-Glutamine, has been shown to induce heat shock protein and protect against vascular hyporeactivity.

It is herein understood by the inventors that inclusion of glutamine in a nutritional composition, will act to increase the production of HSP72, acting as a coactivator, modulating transcriptional regulatory machinery in the promoter region of the gene. Enhanced expression of HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present nutritional composition comprises from about 1 mg to about 1.5 g of glutamine or derivative of glutamine. More preferably, a serving of the present nutritional composition comprises from about 1 mg to about 1.0 g of glutamine or derivative of glutamine. A serving of the present nutritional composition most preferably comprises from about 1 mg to about 750 mg of glutamine or derivative of glutamine. The preferred derivative of glutamine is the dipeptide Alanyl-Glutamine.

Creatine

Creatine is a naturally occurring amino acid derived from the amino acids glycine, arginine, and methionine. Although it is found in meat and fish, it is also synthesized by humans. Creatine is predominantly used as a fuel source in muscle. About 65% of creatine is stored in muscle as phosphocreatine (creatine bound to a Phosphate molecule). Muscular contractions are fueled by the dephosphorylation of adenosine triphosphate (ATP) to produce adenosine diphosphate (ADP) and without a mechanism to replenish ATP stores, the supply of ATP would be rapidly consumed. Phosphocreatine, which is generated from the phosphorylation of creatine by the enzyme Creatine Kinase, serves as a major source of Phosphate from which ADP is regenerated to ATP. As used herein, derivatives of creatine include derivatives such as salts, esters, and amides, as well as other derivatives, including derivatives that become active upon metabolism.

Research indicates that HSP70 is present in an inactive polymerized form that upon stimulation de-polymerized into predominantly more active monomeric form. Of particular significance is that the presence of Creatine Kinase or phosphocreatine contributes to the conversion of polymerized HSP70 to monomeric HSP70.

A specific derivative of creatine, namely creatinol-O-phosphate, has been shown to have beneficial effects in addition to those associated with creatine. Early studies of creatinol-O-phosphate explored its use as a treatment for heart lesions and also its ability to restore reduced cardiac contractile function. As a result of these early studies, creatinol-O-phosphate has been successfully used to improve cardiac parameters in patients with inadequate coronary blood flow. Additionally, early investigators of the properties of creatinol-O-phosphate hypothesized that the cardioprotective effect of creatinol-O-Phosphate was due to action on anaerobic glycolysis. Furthermore, an additional or alternative possible mechanism hypothesized in regard to creatinol-O-phosphate is an effect in the electrophysiological properties of the cell membrane as shown by other investigators.

It is herein understood by the inventors that inclusion of creatine or derivative of creatine in a nutritional composition, will act to increase portion of active HSP70, modulating the de-polymerization of HSP70 by acting as a substrate for Creatine Kinase to support the production of phosphocreatine. Increased monomeric HSP70, will act to increase protein accretion via increased stabilization of nascent proteins. The increased presence of chaperone proteins, i.e. HSP72, in working muscle is important in order to stabilize the large number of new proteins being synthesized by working muscle, leading to increased accumulation of contractile protein, i.e. muscle hypertrophy.

Furthermore, it is herein understood by the inventors that inclusion of creatinol-O-phosphate in a nutritional composition with GGA or a derivative of GGA will, via induction of heat shock proteins (HSP72) and other mechanisms, provide cardioprotective effects.

As used herein, a serving of the present nutritional composition comprises from about 0.5 g to about 5 g of creatine or derivative of creatine. More preferably, a serving of the present nutritional composition comprises from about 1 g to about 3.5 g of creatine or derivative of creatine. A serving of the present nutritional composition most preferably comprises from about 1.5 g to about 3 g of creatine or derivative of creatine.

Alpha Lipoic Acid

Alpha lipoic acid is a co-enzyme found in the cellular energy-producing structures, the mitochondria. Moreover, alpha lipoic acid works in synergy with vitamins C and E as an antioxidant in both water- and fat-soluble environments. As used herein, derivatives of alpha lipoic acid also includes derivatives of alpha lipoic acid such as esters, and amides, as well as other derivatives, including derivatives that become active upon metabolism.

Primarily as an antioxidant, alpha lipoic acid has been demonstrated to have efficacy as a protective against diabetic neuropathy; a benefit mediated by stimulating a heat shock response including HSF-1 and HSP72.

It is herein understood by the inventors that inclusion of alpha lipoic acid or derivatives of alpha lipoic acid in a nutritional composition, will act to increase the expression of heat shock proteins, particularly HSF-1 and HSP72. Enhanced expression of heat shock proteins, particularly HSP72, will act to increase protein accretion via increased stabilization of nascent proteins. The increased expression of chaperone proteins, i.e. HSP72, in working muscle is important in order to stabilize the large number of new proteins being synthesized by working muscle, leading to increased accumulation of contractile protein, i.e. muscle hypertrophy.

As used herein, a serving of the present nutritional composition comprises from about 1 mg to about 250 mg of alpha lipoic acid or derivatives of alpha lipoic acid. More preferably, a serving of the present nutritional composition comprises from about 1 mg to about 100 mg of alpha lipoic acid or derivatives of alpha lipoic acid. A serving of the present nutritional composition most preferably comprises from about 10 mg to about 50 mg of alpha lipoic acid or derivatives of alpha lipoic acid.

In embodiments of the present invention, which are set forth in detail in the examples below, the nutritional composition of the present invention comprises geranylgeranylacetone and glutamine. The nutritional composition is provided in any acceptable and suitable oral dosage form as known in the art. Increased protein accretion is induced and carried out in an individual by administration of the composition of the present invention.

Additional embodiments of the present invention further comprise additional nutraceuticals that are known to function in a manner or through a mechanism such that a heat shock response is induced or in anyway supported. In one such additional embodiment, the nutritional composition of the present invention comprises geranylgeranylacetone, glutamine and creatine or derivatives of creatine. In another such additional embodiment, the nutritional composition of the present invention comprises geranylgeranylacetone, glutamine and alpha lipoic acid or derivatives of alpha lipoic acid.

The nutritional composition of the present invention may be administered in a dosage form having controlled release characteristics, e.g. time-release. Furthermore, the controlled release may be in forms such as a delayed release of active constituents, gradual release of active constituents, or prolonged release of active constituents. Such active constituents release strategies extend the period of bioavailability or target a specific time window for optimal bioavailability. Advantageously the nutritional composition may be administered in the form of a multi-compartment capsule which combines both immediate release and time-release characteristics. Individual components of the nutritional composition may be contained in differential compartments of such a capsule such that the specific components may be released rapidly while others are time-dependently released. Alternatively, a uniform mixture of the various components of the present invention may be divided into both immediate release and time-release compartments to provide a multi-phasic release profile.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a softgel capsule, a tablet, a caplet, or as a dietary gel. The preferred dosage form of the present invention is as a softgel capsule.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art, may be utilized in methods to enhance protein accretion in cells, via increased expression of heat shock proteins, particularly heat shock protein 72 in skeletal muscle, thereby resulting in increased hypertrophy.

Although the following examples illustrate the practice of the present invention in various embodiments, the examples should not be construed as limiting the scope of the invention. Other embodiments will be apparent to one of skill in the art from consideration of the specifications and examples.

### EXAMPLES

### Example 1

A nutritional composition comprising the following ingredients per serving are prepared for consumption as three Softgel Capsules, to be taken twice daily:
from about 1 mg to about 300 mg of geranylgeranylacetone, and from about 1 mg to about 1.5 g of glutamine.

### Example 2

A nutritional composition comprising the following ingredients per serving are prepared for consumption as four Softgel Capsules, to be taken twice daily:
about 50 mg of geranylgeranylacetone, and about 10 mg of glutamine.

### Example 3

A nutritional composition comprising the following ingredients per serving are prepared for consumption as four Softgel Capsules, to be taken twice daily:
about 50 mg of geranylgeranylacetone, 20 mg of glutamine, about 2.0 g of creatine monohydrate and about 100 mg of creatinol-O-phosphate.

### Example 4

A nutritional composition comprising the following ingredients per serving are prepared for consumption as four Softgel Capsules, to be taken twice daily:
about 50 mg of geranylgeranylacetone, about 30 mg of glutamine and about 10 mg of alpha lipoic acid.

### Example 5

A nutritional composition comprising the following ingredients per serving are prepared for consumption as four Softgel Capsules, to be taken twice daily:
about 100 mg of geranylgeranylacetone, about 1 g of glutamine, about 2.5 g of creatine monohydrate, about 25 mg of Schisandrin B, about 100 mg of Ethyl pyruvate, and about 1 mg of Sulbutiamine.

### Example 6

A nutritional composition comprising the following ingredients per serving are prepared for consumption as three Softgel Capsules, to be taken twice daily:
about 10 mg of geranylgeranylacetone, about 50 mg of glutamine, about 10 mg of alanyl-glutamine, and about 75 mg of Ethyl pyruvate.

### Example 7

A nutritional composition comprising the following ingredients per serving are prepared for consumption as three Softgel Capsules, to be taken twice daily:
about 50 mg of geranylgeranylacetone, about 1 mg of glutamine, about 1 mg of paeoniflorin, and about 1 mg of Schisandrin B.

### EXTENSIONS AND ALTERNATIVES

In the foregoing specification, the invention has been described with a specific embodiment thereof; however, it will be evident that various modifications and changes may be made thereto without departing from the broader spirit and scope of the invention.

What is claimed:

1. A nutritional composition for enhancing hypertrophy in skeletal muscle, comprising;
from about 1 mg to about 300 mg of geranylgeranylacetone or derivatives of geranylgeranylacetone; and
from about 1 mg to about 1.5 g of glutamine or derivatives of glutamine.

2. The composition of claim 1, wherein the amount of the geranylgeranylacetone or derivative of geranylgeranylacetone is about 50 mg and the amount of glutamine or derivative of glutamine is about 1 mg.

3. The composition of claim 1, wherein the enhanced hypertrophy in skeletal muscle is due to increased protein accretion.

4. The composition of claim 3, wherein increased protein accretion is facilitated by enhanced expression of heat shock protein.

5. The composition of claim 1, further comprising about 0.5 g to about 5 g of creatine or a derivative of creatine.

6. The composition of claim 1, further comprising about 1 mg to about 250 mg of alpha lipoic acid or derivative of alpha lipoic acid.

7. A method of enhancing hypertrophy in skeletal muscle, comprising the step of administering to a mammal a composition comprising;
from about 1 mg to about 300 mg of geranylgeranylacetone or derivatives of geranylgeranylacetone; and
from about 1 mg to about 1.5 g of glutamine or derivatives of glutamine.

8. The method of claim 7, wherein the amount of the geranylgeranylacetone or derivative of geranylgeranylacetone is about 50 mg and the amount of glutamine or derivative of glutamine is about 1 mg.

9. The method of claim 7, wherein the enhanced hypertrophy in skeletal muscle is due to increased protein accretion.

10. The method of claim 9, wherein increased protein accretion is facilitated by enhanced expression of heat shock protein.

11. The method of claim 7, wherein the composition further comprises about 0.5 g to about 5 g of creatine or a derivative of creatine.

12. The method of claim 7, wherein the composition further comprises about 1 mg to about 250 mg of alpha lipoic acid or derivative of alpha lipoic acid.

13. A nutritional composition for enhancing hypertrophy in skeletal muscle, comprising:
about 50 mg of geranylgeranylacetone; and
about 1 mg of glutamine.

14. The nutritional composition of claim 13, further comprising about 2.5 g of creatine of derivative of creatine.

15. The nutritional composition of claim 14, wherein the creatine derivative is creatinol-O-phosphate.

16. The nutritional composition of claim 13, further comprising about 10 mg of alpha lipoic acid or derivative of alpha lipoic acid.
